(19) United States
(12) Patent Application Publication
(10) Pub. No.: US 2025/0262427 A1
(43) Pub. Date: Aug. 21, 2025

(54) PERCUTANEOUS CIRCULATORY SUPPORT DEVICE WITH IN-VIVO LACTATE SENSOR

(71) Applicant: Boston Scientific Scimed, Inc., Maple Grove, MN (US)

(72) Inventors: Judith Tiferes Wang, Shoreview, MN (US); Jennifer Gibbs, White Bear, MN (US); Michael Kaland, White Bear Lake, MN (US); Zachary Kumor, Plymouth, MI (US)

(73) Assignee: Boston Scientific Scimed, Inc., Maple Grove, MN (US)

(21) Appl. No.: 19/056,996

(22) Filed: Feb. 19, 2025

Related U.S. Application Data
(60) Provisional application No. 63/555,570, filed on Feb. 20, 2024.

(51) Int. Cl. A61M 60/816; A61B 5/00; A61B 5/145; A61B 5/1473; A61M 60/13; A61M 60/174; A61M 60/216; A61M 60/515; A61M 60/865

(52) U.S. Cl. CPC A61M 60/816; A61B 5/14546; A61B 5/1473; A61B 5/6852; A61B 5/742; A61M 60/13; A61M 60/174; A61M 60/216; A61M 60/515; A61M 60/865; A61M 2230/20

(57) ABSTRACT

Example percutaneous circulatory support devices are disclosed. Example percutaneous circulatory support devices includes a catheter including an elongate shaft and a percutaneous blood pump positioned at a distal end of the elongate shaft, the percutaneous blood pump including a blood inlet, a blood outlet, and an impeller assembly positioned therebetween to pump blood from the blood inlet to the blood outlet during use. Further, the example percutaneous circulatory support devices include lactate sensor configured to sense an amount of lactate in the blood.

### CROSS REFERENCE TO RELATED APPLICATIONS

The present application claims the benefit of U.S. Provisional Patent Application Ser. No. 63/555,570, filed Feb. 20, 2024, the disclosure of which is incorporated herein by reference.

### TECHNICAL FIELD

The present disclosure pertains to a catheter including a percutaneous blood pump. More particularly, the present disclosure pertains to a percutaneous circulatory support device with an in-vivo lactate sensor.

### BACKGROUND

Percutaneous mechanical circulatory support devices, such as blood pumps can provide transient support for hours, days or months of use in patients whose heart function or cardiac output is compromised. The percutaneous mechanical circulatory support devices may be sufficiently flexible to be navigated through the vasculature to a patient's heart. For instance, such devices may be delivered percutaneously from the femoral artery, retrograde through the descending aorta, over the aortic arch, through the ascending aorta across the aortic valve, and into the left ventricle.

To assist a clinician using a circulatory support device during a medical procedure, a circulatory support device system may include one or more displays designed to provide easy-to-understand operational and alert information relating to one or more cardiac parameters of the percutaneous circulatory support device, the circulatory support device system and/or the patient. The displayed information may include information sensed by one or more sensors such as sensors which are deployed in-vivo to a patient. However, there is an ongoing need to provide circulatory support device systems including one or more sensors and/or one or more visual displays designed to provide simplified information corresponding to cardiac parameters of a percutaneous circulatory support device and/or a patient.

### BRIEF SUMMARY

This disclosure provides design, material, manufacturing method, and use alternatives for medical devices, including percutaneous circulatory support devices and associated percutaneous blood pumps.

In some aspects, the present disclosure provides a percutaneous circulatory support device, the device comprising a catheter including an elongate shaft and a percutaneous blood pump positioned at a distal end of the elongate shaft, the percutaneous blood pump including a blood inlet, a blood outlet, and an impeller assembly positioned therebetween to pump blood from the blood inlet to the blood outlet during use; and a lactate sensor configured to sense an amount of lactate in the blood.

In some embodiments, which can be used in conjunction with the above aspects, the lactate sensor is an electrochemical sensor.

In some embodiments, which can be used in conjunction with the above aspects and embodiments, the amount of lactate is based on an amount of lactate oxidase (LOD) in the blood.

In some embodiments, which can be used in conjunction with the above aspects and embodiments, the amount of lactate is based on an amount of lactate dehydrogenase (LDH) in the blood.

In some embodiments, which can be used in conjunction with the above aspects and embodiments, the lactate sensor is configured to periodically detect the amount of lactate in the blood.

In some embodiments, which can be used in conjunction with the above aspects and embodiments, the lactate sensor is configured to continuously detect the amount of lactate in the blood.

In some embodiments, which can be used in conjunction with the above aspects and embodiments, the lactate sensor is in direct fluid communication with the blood.

In some embodiments, which can be used in conjunction with the above aspects and embodiments, the lactate sensor is located inside of or on an exterior surface of the percutaneous blood pump.

In some embodiments, which can be used in conjunction with the above aspects and embodiments, the lactate sensor is located between the blood inlet and the blood outlet of the percutaneous blood pump.

In some embodiments, which can be used in conjunction with the above aspects and embodiments, the lactate sensor is located inside of or on an exterior surface of the elongate shaft.

In some embodiments, which can be used in conjunction with the above aspects and embodiments, the lactate sensor is located proximal of the percutaneous blood pump.

In some embodiments, which can be used in conjunction with the above aspects and embodiments, the lactate sensor is mounted in a housing on the elongate shaft with the lactate sensor facing an exterior of the elongate shaft.

In some aspects the present disclosure provides a percutaneous circulatory support system, the system comprising: a percutaneous circulatory support device including: a catheter including an elongate shaft and a percutaneous blood pump positioned at a distal end of the elongate shaft, the percutaneous blood pump including a blood inlet, a blood outlet, and an impeller assembly positioned therebetween to pump blood from the blood inlet to the blood outlet during use in a heart of a patient; and a lactate sensor configured to sense a real-time serum lactate level in the blood of the patient during use of the percutaneous blood pump in the heart of the patient.

In some embodiments, which can be used in conjunction with the above aspects and embodiments, the lactate sensor is secured to an exterior of the elongate shaft proximal of the percutaneous blood pump.

In some embodiments, which can be used in conjunction with the above aspects and embodiments, the lactate sensor is mounted in a housing on the elongate shaft.

In some embodiments, which can be used in conjunction with the above aspects and embodiments, the lactate sensor is positioned between the blood inlet and the blood outlet.

In some embodiments, which can be used in conjunction with the above aspects and embodiments, the system further comprising an introducer sheath, wherein the introducer sheath includes a lumen configured to deliver the percutaneous blood pump to the heart of the patient, and wherein the lactate sensor is secured to the introducer sheath.

In some aspects the present disclosure provides, an introducer sheath for gaining access to a vasculature of a patient comprising: a hub including a hemostasis valve; an elongate shaft extending distally from the hub, the elongate shaft including a lumen extending therethrough; and a lactate sensor configured to sense an amount of lactate in the blood.

In some embodiments, which can be used in conjunction with the above aspects and embodiments, the lactate sensor is mounted on a distal end region of the elongate shaft.

In some embodiments, which can be used in conjunction with the above aspects and embodiments, the lactate sensor is movably postionable through a passageway of the hub.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump with a lactate sensor;

FIG. 2 shows the distal end region of the percutaneous circulatory support device of FIG. 1 including the percutaneous blood pump with the lactate sensor;

FIG. 3A is a side view of a portion of the percutaneous blood pump of FIG. 2, with a lactate sensor;

FIG. 3B is a cross-sectional view of the portion of the percutaneous blood pump of FIG. 3A, with a lactate sensor;

FIG. 3C is a cross-sectional view of a portion of the flexible cannula of the percutaneous blood pump of FIG. 2, with a lactate sensor therein;

FIG. 4 is a perspective view of an exemplary introducer sheath;

FIG. 5 a cross-sectional view of the hub of the introducer sheath of FIG. 4, illustrating a lactate sensor inserted into the insertion sheath;

FIG. 6 illustrates a view of a lactate sensor positioned proximate a distal end of the elongate shaft of the insertion sheath of FIG. 4;

FIG. 7 illustrates an example percutaneous circulatory support system including a percutaneous blood pump with a lactate sensor positioned in the heart of a patient.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

A serum lactate level in a patient is an important biomarker that may be used to monitor tissue perfusion in the patient. For instance, lactic acidosis/hyperlactatemia is a prognostic factor in patients with acute myocardial infarction and cardiogenic shock. Current approaches for measuring a serum lactate level entail drawing a blood sample from a patient and transporting the blood sample to a lab for analysis (e.g., for measurement of a serum lactate level of the blood sample). However, such approaches may be costly, time-consuming, and/or do not reflect a real-time serum lactate level in the patient. For instance, such approaches may impart a large amount of delay between a time that the blood sample is drawn and a time when a serum lactate level (e.g., as measured by the lab) is conveyed to a clinician (e.g., doctor). Thus, any intervention (e.g., removal of a pump, alteration of a pump setting, etc.) performed by the clinician based on the serum lactate level may be delayed and/or is not based on real-time information. Further, any change in serum lactate level of the patient that occurs after the blood sample is drawn is not accounted for.

Accordingly, this disclosure is directed to percutaneous circulatory support devices and percutaneous circulatory support systems that include a lactate sensor. Notably, the lactate sensor can provide real-time (e.g., continuous) in-vivo serum lactate measurements. The real-time in-vivo serum lactate measurements can be conveyed in real-time to a clinician. Thus, the devices and systems herein permit timely monitoring and, when needed, timely patient interventions (e.g., escalation or de-escalation of cardiac support therapy), unlike other approaches such as those that rely on separate laboratory testing and/or the addition of extraction reagents to a blood sample outside of a patient.

Additionally, the lactate sensor may be in direct contact with blood in-vivo a patient. As such, the devices and systems herein can provide accurate serum lactate levels, in contrast to other approaches such as those that seek to measure serum lactate levels indirectly (e.g., via skin/sweat measurement and/or via a subcutaneous patch) and therefore may be relatively inaccurate (e.g., may inaccurately measure an amount of lactate due to deployment issues/positioning variation and/or variation associated with changes in temperature and/or pH).

Moreover, in some embodiments, the in-vivo lactate measurements may be made continuously while the patient is being treated with a percutaneous circulatory support device. That is, it may be desirable to continuously measure an individual's lactate levels via an implanted lactate sensor while a percutaneous circulatory support device is in operation within the patient. For example, continuous (or near continuous) in vivo-lactate monitoring may be beneficial for people with chronic, slowly changing lactate levels and thereby permit graphing or other methods of determining a trend in the serum lactate levels over time during the operation of a percutaneous circulatory support device. In addition, continuous (or near continuous) in vivo-lactate monitoring may be beneficial for people and/or patients that may be prone to abrupt sizable changes in lactate levels (e.g., due to accelerated degradation of heart tissue). Further, such continuous monitoring of lactate levels may mitigate the pain and/or the expense that is typically associated with multiple blood draws to measure lactate levels via multiple laboratory assays.

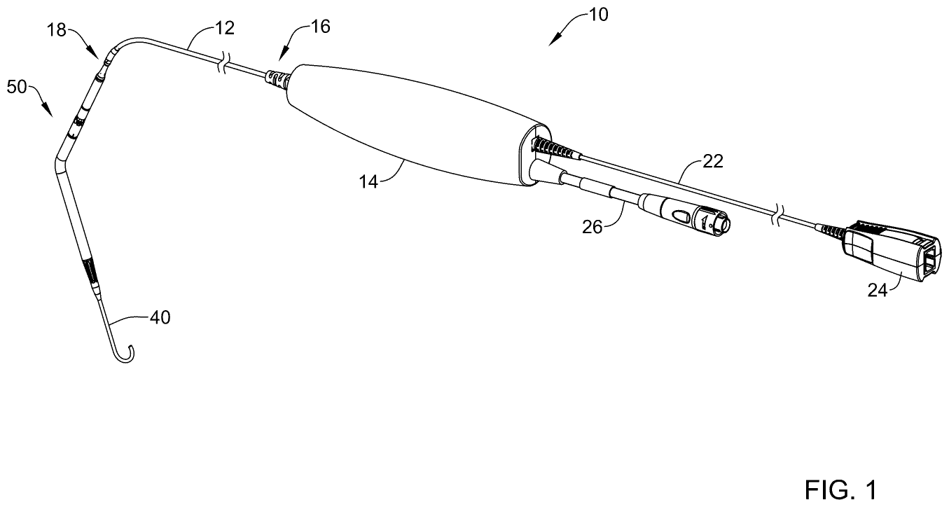

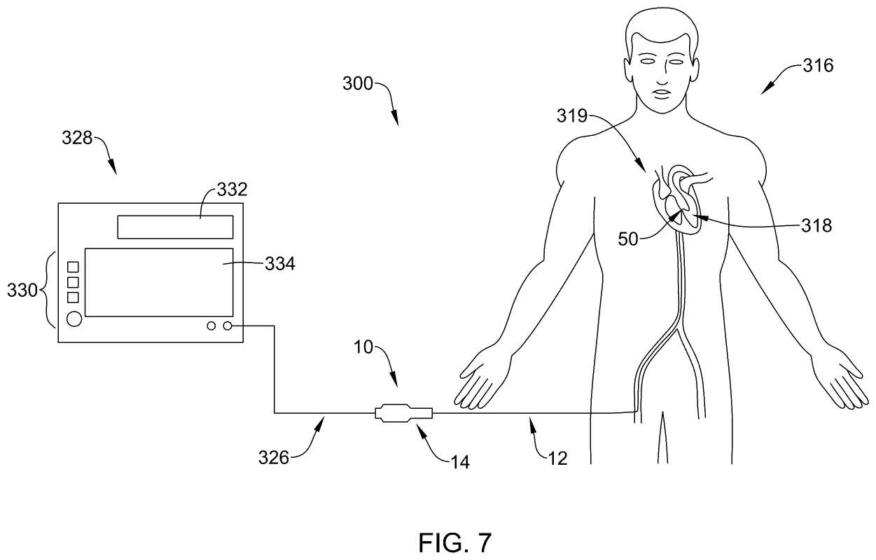

FIG. 1 is a perspective view of an exemplary percutaneous circulatory support device (e.g., catheter) 10 including a percutaneous blood pump 50 with a lactate sensor (not shown in FIG. 1). As illustrated in FIG. 1, the percutaneous blood pump 50 may be located at a distal end region of the catheter 10. The catheter 10 may be coupled to or include the percutaneous blood pump 50, with an elongate shaft 12 of the catheter 10 extending proximally from the percutaneous blood pump 50 and a distal tip 40 extending distally from the percutaneous blood pump 50. For instance, a proximal end 16 of the elongate shaft 12 may be coupled to a control module 14 and a distal end 18 of the elongate shaft 12 may be coupled to the percutaneous blood pump 50. An electrical cable 22 may extend from the control module 14 to a connector 24 at a proximal end thereof. The connector 24 may be configured to be connected to a controller or console (not shown in FIG. 1) for controlling the percutaneous blood pump 50, such as providing electrical power to the blood pump 50. The catheter 10 may also include an extension 26 connectable to the controller or console (not shown in FIG. 1) for sending and/or receiving signals, such as from one or more sensors during operation of the percutaneous blood pump 50. In some instances, the electrical power cable and/or sensor cables may be combined in a single cable harness, if desired. For instance, as detailed with respect to FIG. 7, a console may include one or more displays that are configured to display information such as a real-time in-vivo lactate measurement measured be a lactate sensor, as described herein.

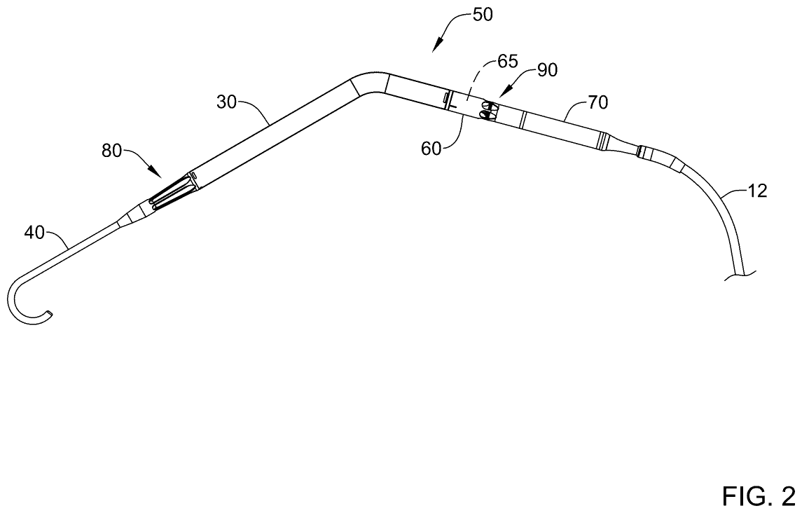

Additional features of the percutaneous blood pump 50 are illustrated in FIG. 2. The percutaneous blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller secured to an impeller shaft that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the percutaneous blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the percutaneous blood pump 50 (e.g., the flexible cannula 30). In some instances, the blood outlet 90 may include a plurality of blood outflow windows arranged around a circumference of the impeller housing 60. In other embodiments, the blood inlet 80 and/or the blood outlet 90 may be formed on other portions of the percutaneous blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

In some embodiments, the system herein includes a lactate sensor provided with the percutaneous circulatory support device 10 and/or provided with an introducer sheath configured to be used with introducing and/or maintaining the percutaneous circulatory support device 10 in a patient. Accordingly, in some embodiments, the lactate sensor can be located on an exterior (e.g., outer) surface of the percutaneous circulatory support device 10 (e.g., an exterior surface of the elongate shaft 12, an exterior surface of the cannula 30, an exterior surface of the motor housing 70, or an exterior surface of the impeller housing 60) and/or on an exterior surface of an introducer sheath, as described herein. However, in some embodiments the lactate sensors herein can be configured (e.g., sized, shaped, etc.) to be disposed within a lumen (e.g., on an inner surface of the cannula 30) of the percutaneous circulatory support device 10 or within a lumen of an introducer sheath, as described herein.

In some embodiments, the lactate sensor may be an electrochemical sensor, namely an electrochemical lactate sensor. The lactate sensor may be configured to detect an in-vivo lactate level using coulometric, amperometric, voltammetric, and/or potentiometric electrochemical detection techniques. For instance, the lactate sensor may include a lactate-responsive analyte sensor such as a lactate-responsive-enzyme based analyte sensor. In some embodiments, the lactate sensor may be configured to detect lactate oxidase and/or lactate dehydrogenase in-vivo a patient. In some embodiments, the lactate sensor may be configured to detect lactate oxidase in-vivo a patient. In some embodiments, the lactate sensor may be configured to detect lactate dehydrogenase in-vivo a patient. In some embodiments, the lactate sensor can include a plurality of electrodes such as two-electrodes or three-electrodes. In other embodiments, the lactate sensor can include another quantity of electrodes, if desired. As is conventionally known, a two-electrode configuration may employ a first working electrode and a second electrode that functions as both a counter electrode and a reference electrode, while a three-electrode configuration may employ a working electrode, a counter electrode, and a reference electrode. In some embodiments, an electron transfer material can be present in the lactate sensors herein. A suitable electron transfer agent can facilitate the transfer of electrons to or from the working electrode when an analyte (enzyme substrate) such as lactic acid undergoes a redox reaction. In some embodiments, the lactate sensor can include a wireless transmitter configured to wirelessly transfer information (e.g., to a controller or computing device remotely located from the lactate sensor). For instance, the information can include information indicative of an in-vivo lactate level detected by the lactate sensor. In other embodiments, the lactate sensor can be wired to a controller or computing device for transmitting information indicative of an in-vivo lactate level detected by the lactate sensor.

In some instances, the lactate sensor can be non-removably coupled to a surface such as an exterior surface of the percutaneous circulatory support device 10 (e.g., an exterior surface of the elongate shaft 12, an exterior surface of the cannula 30, an exterior surface of the motor housing 70, or an exterior surface of the impeller housing 60) or an inner surface of a lumen of the percutaneous blood pump 50 (e.g., the lumen of the cannula 30). Having the lactate sensor be non-removably coupled to the surface can ensure that the lactate sensor does not inadvertently become dislodged from the surface (e.g., an inner surface or exterior surface of the percutaneous circulatory support device 10) and/or may promote other aspects herein such as providing an accurate in-vivo lactate measurement. However, in some instances, the lactate sensor can be removably coupled to a surface such as an exterior surface of the percutaneous circulatory support device 10 (e.g., an exterior surface of the elongate shaft 12, an exterior surface of the cannula 30, an exterior surface of the motor housing 70, or an exterior surface of the impeller housing 60) or an inner surface of a lumen of the percutaneous blood pump 50 (e.g., the lumen of the cannula 30). Having the lactate sensor be removably coupled to the surface can permit the lactate sensor to be readily changed or cleaned (e.g., responsive to a malfunction of a lactate sensor, fouling of the lactate sensor, etc.), and thereby can promote aspects herein such as providing accurate in-vivo lactate measurements.

While generally described herein as an individual lactate sensor, in some embodiments a plurality of lactate sensors may be employed with the percutaneous circulatory support device 10 and/or introducer sheath. For instance, a plurality of lactate sensors may be employed in-vivo a patient, as described herein, to determine respective in-vivo lactate levels at different locations in a patient and/or to determine an average in-vivo lactate level.

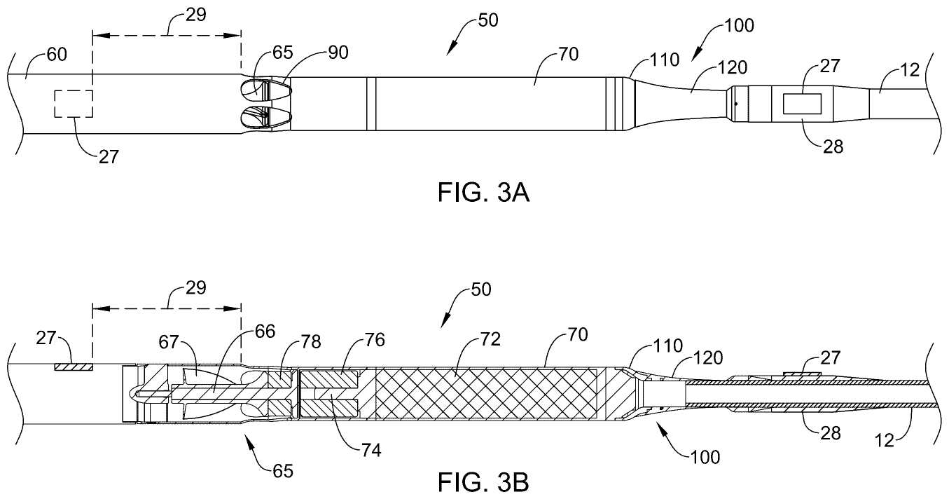

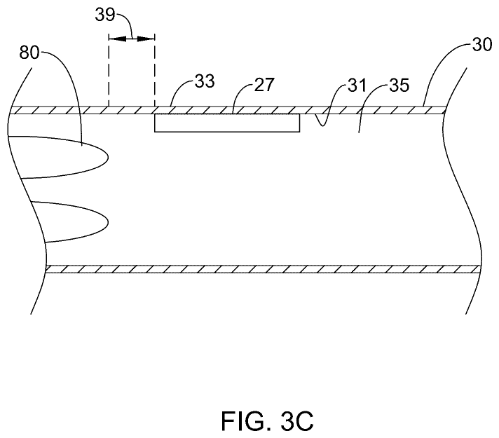

FIG. 3A is a side view of a portion of the percutaneous blood pump of FIG. 2. As shown in FIG. 3A, the percutaneous circulatory support device 10 including the percutaneous blood pump 50 can be connected to the elongate shaft 12 at a junction 100. The junction 100 may be configured to mechanically connect the motor housing 70 (e.g., a metallic motor housing) of the blood pump 50, or other component of the blood pump 50, to the distal end region of the elongate shaft 12 (e.g., a polymeric tubular member). A proximal end of the impeller of the impeller assembly 65 is also visible through the outflow windows of the blood outlet 90.

The junction 100 may include an end cap 110 having a proximal end region surrounding the distal end region of the elongate shaft 12, and a fillet of material 120 surrounding the proximal end region of the end cap 110 and extending proximally therefrom. The fillet of material 120 may extend proximal of the end cap 110 and surround a portion of the elongate shaft 12 extending proximal of the end cap 110, among other possible configurations.

FIG. 3B is a cross-sectional view of the portion of the percutaneous blood pump of FIG. 2. As shown in FIG. 3B, the impeller assembly 65 includes a drive shaft 66 and an impeller 67 coupled thereto, where the drive shaft 66 is configured to rotate with the impeller 67. As shown, the drive shaft 66 is at least partially disposed within the impeller 67. The impeller assembly 65 further includes a driven magnet 78 coupled to, and at least partially surrounding, the drive shaft 66 and/or the impeller 67. The driven magnet 78 may be any type of magnetic rotor capable of being driven by a driving magnet assembly. In this manner, as a magnetic field is applied to the driven magnet 78 by the driving magnet assembly, the driven magnet 78 rotates, causing the drive shaft 66 and impeller 67 to rotate.

As shown in FIG. 3B, the driving magnet assembly includes a driving magnet 76 coupled to a drive line 74 configured to transfer torque from a motor 72 to the driving magnet 76. The motor housing 70 may be configured to hermetically seal the driving magnet 76 and the motor within the motor housing 70, and thus fluidly isolate the driving magnet 76 from the driven magnet 78. Electrical wires, not shown, may extend through a lumen of the elongate shaft 12 to provide electrical power to the motor 72. In other instances, the drive line 74 may be directly coupled to the impeller 67 and/or drive shaft 66, or other driven trains may be provided to transfer torque from the motor 72 to the impeller 67.

In some embodiments, a lactate sensor 27 can be included on an exterior surface or outer surface of the percutaneous circulatory support device 10. FIGS. 3A and 3B show several possible locations of a lactate sensor 27 on the elongate shaft 12. The intent is to show the possible locations, but not necessarily to indicate that more than one lactate sensor 27 is needed in embodiments of this disclosure, unless described otherwise herein. For instance, as shown in FIGS. 3A and 3B, a lactate sensor 27 can be disposed on an exterior surface of the elongate shaft 12 proximal of the percutaneous blood pump 50, for example, proximal of the junction 100. In some instances, the lactate sensor 27 may be indirectly secured to the elongate shaft 12 in a housing 28, with the housing 28 in turn secured to the elongate shaft 12. In other instances, the lactate sensor 27 may be directly secured to the elongate shaft 12. While FIG. 3A illustrates the lactate sensor 27 as being on an exterior surface of the elongate shaft 12, the present disclosure is not so limited. In other instances, the lactate sensor 27 can be disposed on an exterior surface of the motor housing 70, the impeller housing 60, or the cannula 30, if desired. Signal wires may extend from the lactate sensor 27 through a lumen of the elongate shaft 12 to a console or other controller configured to collect, store and/or display data from the lactate sensor 27. In some embodiments the lactate sensor 27 can be disposed on a different exterior surface of the percutaneous circulatory support device 10, such as on a different exterior surface of the percutaneous blood pump 50 or elongate shaft 12. Having the lactate sensor 27 be disposed on an exterior surface may permit the lactate sensor 27 to be readily exposed to blood flowing past the lactate sensor and/or permitting the lactate sensor 27 to be cleaned or replaced and/or may otherwise promote aspects.

In some embodiments, the lactate sensor 27 may be secured to the elongate shaft 12 at a location that is at least a threshold distance from the percutaneous blood pump 50. The threshold distance from the percutaneous blood pump 50 can be a value in a range from about 1.0 centimeters to about 30.0 centimeters. Having the lactate sensor 27 disposed on the elongate shaft 12 at least the threshold distance proximal of the percutaneous blood pump 50 can mitigate or eliminate an amount of turbulence or irregularity in flow of the blood that may otherwise be due to a proximity of the lactate sensor 27 to the percutaneous blood pump 50. The lactate sensor 27 may be located distal of the control module 14 such that the lactate sensor 27 is positioned within the patient's vasculature when the percutaneous blood pump 50 is positioned across the aortic valve of the patient's heart. For example, the lactate sensor 27 may be a distance from the control module 14 of about 100 centimeters or more, about 80 centimeters or more, or about 50 centimeters or more.

Additionally or alternatively, a lactate sensor 27 can be included on an interior surface or inner surface of the percutaneous circulatory support device 10. That is, in some embodiments the lactate sensor 27 can be included within the interior of a component of the percutaneous circulatory support device 10. For instance, the lactate sensor 27 can be included in a lumen extending through at least a portion of the percutaneous blood pump 50 in contact with blood flowing therein. As illustrated in FIGS. 3A and 3B, the lactate sensor 27 may be located in a lumen in the percutaneous blood pump 50 at a first location between the blood inlet 80 and the blood outlet 90. Specifically, the lactate sensor 27 may be located within the interior of the impeller housing 60, such as secured to an inner surface of the impeller housing 60, such as distal of the blood outlet 90. In other instances, the lactate sensor 27 may be located within the interior of the cannula 30, such as secured to an inner surface of the cannula 30, at a location between the blood inlet 80 and the blood outlet 90. In some instances, the location of the lactate sensor 27 may correspond to a location between the blood inlet 80 and the blood outlet 90 and that is relatively proximate to the blood outlet 90, as illustrated in FIG. 3B.

FIG. 3C is a cross-sectional view of a portion of the flexible cannula 30, with a lactate sensor 27 positioned within the interior of the flexible cannula 30. That is, the lactate sensor 27 may be positioned on an inner surface 31 (opposite the exterior surface 33) facing a lumen 35 extending through the flexible cannula 30, among other possibilities. The location of the lactate sensor 27 in FIG. 3C may correspond to a location between the blood inlet 80 and the blood outlet 90 that is relatively proximate to the blood inlet 80. Thus, blood flowing through the lumen 35 of the flexible cannula between the blood inlet 80 and the blood outlet 90 may flow past and be in contact with the lactate sensor 27.

Having the lactate sensor 27 disposed within the lumen of the percutaneous blood pump 50 may permit the lactate sensor 27 to directly contact blood flowing through the percutaneous blood pump 50 and/or permit accurate lactate measurements at least due to the proximity of the lactate sensor 27 relative to a heart of the patient.

In some embodiments, the lactate sensor 27 may be located on a surface (e.g., a surface in a lumen) of the percutaneous blood pump 50 at a location that is at least a threshold distance 39 from the blood inlet 80 and/or is at least a threshold distance 29 from the blood outlet 90. The threshold distance 39 from the blood inlet 80 may be a value in a range from about 1.0 to about 5.0 centimeters, among other possibilities. Similarly, the threshold distance 29 from the blood outlet 90 may be a value in a range from about 1.0 to about 5.0 centimeters, among other possibilities. Having the lactate sensor 27 be located at a location that is a threshold distance 39 (FIG. 3C) from the blood inlet 80 and/or is a threshold distance 29 (FIGS. 3A and 3B) from the blood outlet 90 may promote aspects herein such as promoting accurate in-vivo lactate measurements of blood in the percutaneous blood pump 50. For instance, locating the lactate sensor at least the threshold distance 39 away from the blood inlet 80 and/or at least the threshold distance 29 from the blood outlet 90 may mitigate an amount of turbulence or irregularity in flow of the blood, as compared to locations that are more proximate to the blood inlet 80 and/or the blood outlet 90. Furthermore, locating the lactate sensor proximal of the blood outlet 90 (as shown with the lactate sensor 27 placed on the exterior of the elongate shaft 12 proximal of the percutaneous blood pump 50) may also mitigate an amount of turbulence or irregularity in flow of the blood passing the lactate sensor 27.

While in some embodiments the lactate sensor 27 is described as being located in a lumen in the percutaneous blood pump 50, as mentioned the disclosure is not so limited. Rather, in some embodiments, the lactate sensor 27 may be located on exterior surface of the percutaneous blood pump 50, as illustrated in FIG. 3A, or may be located outside of the percutaneous circulatory support device 10. For example, the lactate sensor may be located in (e.g., on an interior surface) or on (e.g., on an exterior surface) the elongate shaft 12 at a location that is proximal or distal to the percutaneous blood pump 50. In some instances, the lactate sensor 27 may be located in a lumen extending through the elongate shaft 12 at a location between the percutaneous blood pump 50 and a control module 14 that is coupled to a proximal end of the elongate shaft 12.

In other instances, the lactate sensor 27 may be located within a lumen of the elongate shaft 12 (not shown), if desired. That is, the lactate sensor 27 may be located in the elongate shaft 12 between the percutaneous blood pump 50 and the control module 14. Having the lactate sensor 27 be located in a lumen in the elongate shaft 12 may mitigate an amount of turbulence or irregularity in flow of the blood may be reduced as compared to locations that are more proximate to the percutaneous blood pump 50.

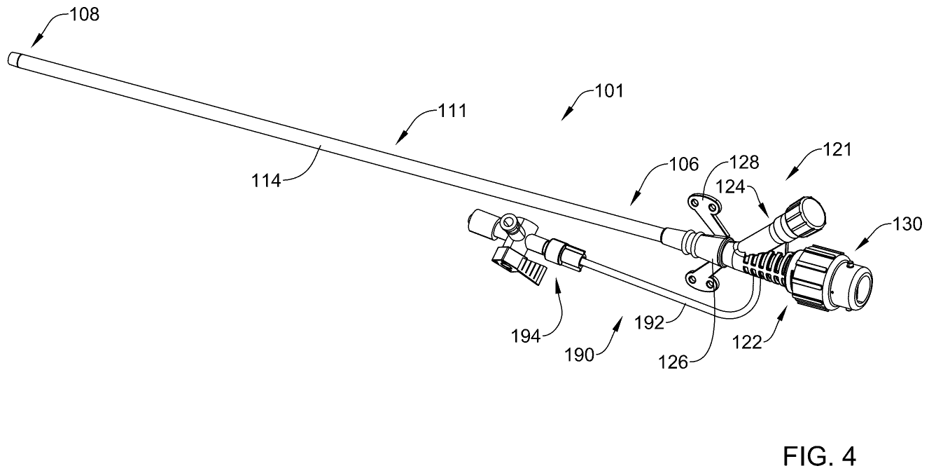

Moreover, in some embodiments the lactate sensor 27 may be provided with a device or instrument other than the percutaneous circulatory support device 10. For instance, the lactate sensor 27 may be provided with an introducer sheath configured to be used in conjunction with the percutaneous circulatory support device 10. FIG. 4 is a perspective view of an introducer sheath 101 for use in providing vascular access for introducing the percutaneous blood pump of FIG. 1 into a vasculature at a vascular access site. The introducer sheath 101 may include a hub 121, as well as an elongate shaft 114 extending distally from the hub 121 to a distal end region 108 and defining the body portion 111 of the introducer sheath 101. The introducer sheath 101 may also include a flush line 190 extending from the hub 121. The flush line 190 may include a tubular member 192 extending from the hub 121 and in fluid communication with a lumen of the hub 121. The tubular member 192 may extend to a stopcock 194, such as a three-way stopcock.

The hub 121 may also include a strain relief 126 configured to provide a transition in flexibility along the proximal end region 106 of the elongate shaft 114. The strain relief 126 may include a body attached to a main body of the hub 121, as will be described further herein. The strain relief 126 may include one or more suture pads 128 configured to facilitate securing the hub 121 against the patient once the introducer sheath 101 has been positioned in the blood vessel of the patient.

The hub 121 may include a main port 122 and a side port 124 extending from the main port 122. In some instances, the side port 124 may extend at an acute angle from the main port 122. The main port 122 and/or the side port 124 may provide access to one or more lumens extending through the body portion 111 (e.g., through the elongate shaft 114) of the introducer sheath 101. In some instances, the main port 122 may be a tightening port 130.

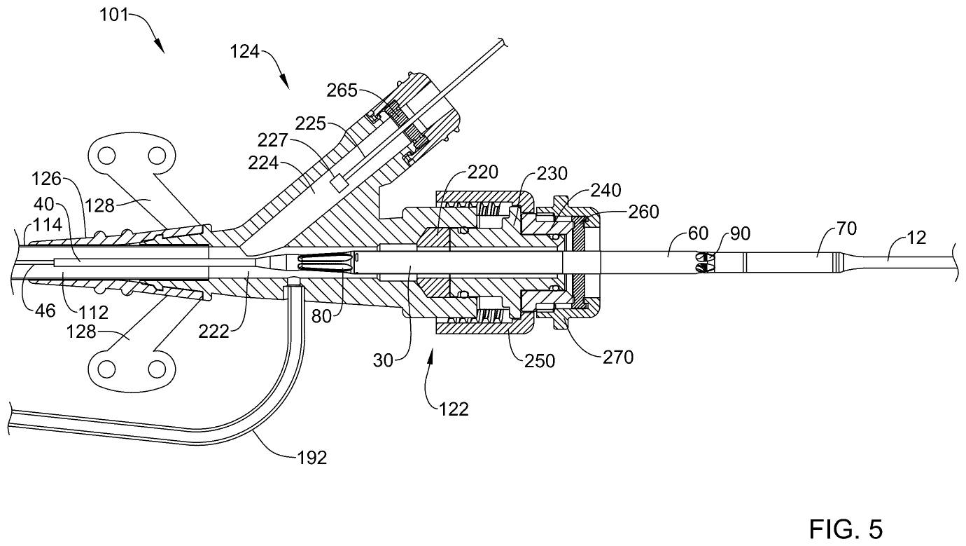

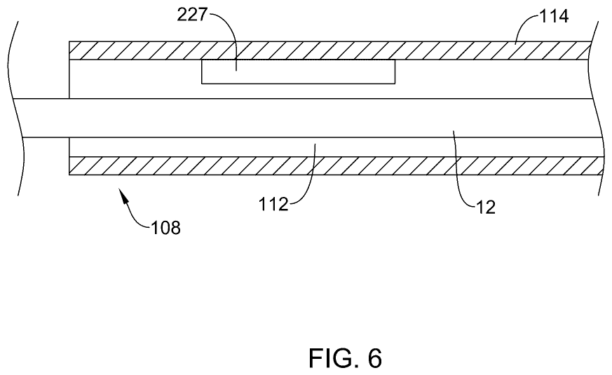

Turning to the cross-sectional view of the hub 121, shown in FIG. 5, the side port 124 may include a passage 224 extending therethrough converging with a passage 222 of the main port 122. The passage 222 and/or the passage 224 may be in fluid communication with the lumen 112 of the introducer sheath 101 extending to a distal opening of the elongate shaft 114. An elastomeric seal 265 may be positioned along the passage 224 of the side port 124. The elastomeric seal 265 may be a slit valve (e.g., a cross-slit valve), a dome valve, a duckbill valve, or any other desired valve configured to seal around an elongate shaft of a medical device when passed therethrough. In some instances, the elastomeric seal 265 may include one or more slits (e.g., crossing slits) extending entirely through the seal wall and/or one or more slits (e.g., crossing slits) extending only partially through the seal wall. For instance, the elastomeric seal 265 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seal 265 may be formed of any desired flexible material, such as silicone, polyurethane, etc.

The main port 122 may include a primary seal and a secondary seal spaced apart from the primary seal along a length of the main port 122. The components of the main port 122 may form a hemostasis valve. For example, the main port 122 may include a compressible seal (e.g., Tuohy seal) 220 and an elastomeric seal 260 spaced apart from the compressible seal 220. The main port 122, which may be considered a tightening port, may further include a pusher 230, a holder 240, a lock nut 250, and/or a main port lid 270. Rotation of the lock nut 250 may actuate the pusher 230 toward/away from the compressible seal 220 to adjust the size of the opening through the compressible seal 220. The compressible seal 220 may be movable between an open state, allowing a medical device to pass through the opening, and a closed state, sealing the compressible seal 220 around the medical device. The compressible seal 220 may be formed of any desired flexible material, such as silicone, polyurethane, etc.

In use, the percutaneous blood pump 50 of the percutaneous circulatory support device 10 may be inserted through the introducer sheath 101 along a guidewire 46. For example, the percutaneous blood pump 50 may be advanced through the main port 122 and into the lumen of the elongate shaft 114 of the introducer sheath 101. The elastomeric seal 260 may seal around an outer perimeter of the cannula 30 of the percutaneous blood pump 50 and substantially prevent blood from escaping from the main port 122.

The elongate shaft 12 of the percutaneous circulatory support device 10 may be further advanced through the introducer sheath 101 into the body of the patient to position the percutaneous blood pump 50 in a desired location, such as across the aortic valve between the left ventricle of the heart and the aorta. Once the percutaneous blood pump 50 is positioned at a desired location within the vasculature of the patient (e.g., within the heart of the patient), the compressible seal 220 may be compressed or tightened around the perimeter of the elongate shaft 12 of the catheter 10 to prevent blood from leaking past the elongate shaft 12 and out of the main port 122 and/or lock the elongate shaft 12 of the catheter 10 from axial movement relative to the hub 121.

As illustrated in FIG. 5, in some instances a lactate sensor 227 may be insertable into the side port 124 of the introducer sheath 101 for use with the percutaneous circulatory support device 10 (positioned in the main port 122). For instance, the lactate sensor 227 may be included with a catheter 225, which can be inserted in the side port 124 and removable therefrom. The lactate sensor 227 can be inserted a distance into the passage 224 of the side port 124 to place the lactate sensor 227 in contact with blood. In some instances, the lactate sensor 227 may be maintained in the passage 224 of the side port 124. However, in other instances, the lactate sensor 227, mounted on the catheter 225 may be advanced distally though the lumen 112 of the elongate shaft 114 of the introducer sheath 101, such as along an exterior of the elongate shaft 12 of the percutaneous circulatory support device 10 disposed therein. In some instances, the lactate sensor 227 may be positioned within the lumen 112 of the elongate shaft 114. However, in other instances, the lactate sensor 227 may be advanced distally beyond the elongate shaft 114 to a location within the patient's vasculature beyond the elongate shaft 114.

In other embodiments the lactate sensor 227 may be secured to the introducer sheath 101. For example, FIG. 6 is a cross-sectional view of a distal end region of the elongate shaft 114 of the introducer sheath 101, illustrating the lactate sensor 227 secured within the lumen 112 of the insertion sheath proximate the distal end region 108 thereof. Signal wires may extend from the lactate sensor 227 through a lumen of the elongate shaft 114 to a console or other controller configured to collect, store and/or display data from the lactate sensor 227. The elongate shaft 12 of the percutaneous circulatory support device 10 may extend through the lumen 112 of the elongate shaft 114 along the lactate sensor 227. In other instances, the lactate sensor 227 may be secured to and open to an exterior of the elongate shaft 114, such as on the exterior of the elongate shaft 114 proximate the distal end region 108, or at the distal tip of the elongate shaft 114, for example.

FIG. 7 illustrates an example percutaneous circulatory support system 300 including a console 328, a percutaneous circulatory support device 10 including an elongate shaft 12 and a percutaneous blood pump 50 positioned at a distal end of the elongate shaft 12. As shown in FIG. 7, the percutaneous blood pump 50 can be positioned in the heart 319 of the patient 316. In some embodiments, the lactate sensor (not shown in FIG. 7) can be positioned in-vivo to the patient 316. For instance, the lactate sensor can be positioned in a vasculature of the patient 316 and/or positioned in the heart 319 of the patient 316.

The elongate catheter shaft 12 (e.g., a flexible elongate catheter shaft) may have a first end attached to a control module 14 and a second end attached to a percutaneous blood pump 50. FIG. 7 illustrates the percutaneous blood pump 50 positioned in the left ventricle 318 of the patient 316. The percutaneous blood pump 50 may be delivered (e.g., tracked) to the left ventricle 318 percutaneously over a guidewire. For example, the catheter shaft 12 and blood pump 50 may be tracked over a guidewire through the femoral artery and the descending aorta, over the aortic arch, through the ascending aorta, past the aortic valve and into the left ventricle 318.

In some examples, the catheter shaft 12 of the percutaneous circulatory support device 10 may include one or more blood inlets located on a distal region of the catheter shaft 12, and one or more blood outlets which are along a housing of the percutaneous blood pump 50. In some examples, the percutaneous blood pump 50 may be positioned within the heart 319 such that the one or more blood inlets positioned along the distal region of the catheter shaft 12 may be positioned in the left ventricle 318 and the one or more blood outlets located along the housing of the percutaneous blood pump 50 may be positioned in the aorta. Additionally, the percutaneous blood pump 50 may include an electrically powered motor that drives rotation of an impeller (positioned within the housing of the blood pump 50). In some examples, the motor may power the rotation of the impeller via electromagnetic induction. The spinning impeller may draw blood from the left ventricle (via the one or more blood inlets located on a distal region of the catheter shaft 12) into the aorta (via the one or more outflow inlets located along the housing of the blood pump 50). In other words, an electrically powered motor drives the impeller to pump blood from the left ventricle through the aortic valve and into the ascending aorta.

FIG. 7 further illustrates that a first end of the catheter shaft 12 may be attached to a control module 14. The control module 14 may include a distal end region attached to the catheter shaft 12 and a proximal end region attached to an electrical power cable 326. The electrical power cable 326 may include an end region connected to a console 328. It can be appreciated that the control module 14 may include one or more actuators (e.g., buttons, levers, dials, switches, etc.) designed to permit a clinician to control various functions of the percutaneous blood pump 50. For example, a clinician may be able to control the speed of the motor and/or the impeller located in the percutaneous blood pump 50 via actuation of one or more actuators located on the control module 14. In some embodiments, the console may include a display a visual representation of the real-time amount of serum lactate

Additionally, the percutaneous circulatory support system 300 may include one or more sensors positioned along the console 328, the control module 14, the catheter shaft 12 and/or the percutaneous blood pump 50. The one or more sensors positioned within the console 328, the control module 14, along the catheter shaft 12 and/or the percutaneous blood pump 50 may be designed to monitor blood pressures (e.g., arterial pressure, venous pressure), blood velocity, blood flowrate, blood lactate levels, or other parameters. Additionally, the one or more sensors positioned along the console 328, within the control module 14, the catheter shaft 12 and/or the percutaneous blood pump 50 may be designed to monitor other parameters of the percutaneous circulatory support system 300, the catheter 10 and/or the patient 316.

Example parameters that may be sensed and monitored may include the electrical connection of the electrical cable 326 to the console 328, an optical and/or electrical connection to a pressure sensor positioned in the catheter shaft 12 and/or the percutaneous blood pump 50, a power current to the percutaneous blood pump 50, the speed of the impeller of the percutaneous blood pump 50, an arterial pressure, a venous pressure, a power current to the motor, the speed of the motor, an in-vivo lactate measurement (such as from the lactate sensor 27/227), rotations per minute measurement of a motor, the voltage driving the motor, the back-EMF from the motor, etc. and any combination or temporal pattern of signals corresponding to one or more of the parameters listed herein. Further, additional parameters (e.g., flow through the percutaneous blood pump 50) may be derived by processing combinations of any signal listed herein in a time dependent manner.

Additionally, FIG. 7 illustrates that the console 328 may include one or more control knobs (e.g., buttons, knobs, dials, etc.) 330 and/or one or more displays. For example, FIG. 7 illustrates the console 328 may include a first display 332 and a second display 334. It can be appreciated that the console 328 may include more than two displays. Additionally, while FIG. 7 illustrates the first display 332 and the second display 334 integrated into the console 328, it is contemplated that the percutaneous circulatory support system 300 may be designed such that the first display 332, the second display 334 or both the first display 332 and the second display 334 are separate, distinct components of the catheter 10. In other words, the first display 332, the second display 334 or both the first display 332 and the second display 334 may be separate stand-alone displays, apart from the console 328. In some examples, the first display 332 and the second display 334 may get their respective data from separate sources.

In some examples, the second display 334 may be designed to attach to the console 328 and/or the first display 332. For example, the first display 332 may be integrated into the console 328 while the second display 334 may be configured to attach to portion of the console 328. In yet other examples, both the first display 332 and the second display 334 may be a separate stand-alone display whereby the second display 334 may be configured to attach to the first display 332, or wherein the first display 332 may be configured to attach to the second display 334.

The console 328 may include, among other suitable components, one or more processors, memory, and an I/O unit. The processor of the console 328 may include a single processor or more than one processor working individually or with one another. The processor may be configured to execute instructions, including instructions that may be loaded into the memory and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor of the console may be configured to execute program instructions. Program instructions may include, for example, firmware, microcode or application code that is executed by the processor, a microprocessor and/or microcontroller. The one or more processors may be configured to each manage different functions. They may also be configured to concurrently perform the same functions (e.g., redundant system). Further yet, they may be configured such that a first processor performs a given function and second processor checks the result of the function of the first processor for correctness (e.g., command-monitor system).

In some examples, the first display 332 may controlled primarily by the console's firmware control instructions and, therefore, may require relatively little processing power, relatively few instructions and very simple communication between the processor and the first display 332, compared to the second display 334 (e.g., a touch screen display 34), which may be controlled primarily by an embedded computer with a flexible and relatively complex communication protocol.

The memory of the console 328 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory may be or may include a non-transitory computer readable medium.

The I/O units of the console 328 may include a single I/O component or more than one I/O component each working individually or with one another. Example I/O units may be any type of communication port configured to communicate with other components of the percutaneous circulatory support system 300. Example types of I/O units may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

The console 328 may be configured to collect, record, monitor, save, evaluate and/or display data relevant to the lactate level in the blood sensed by the lactate sensor 27/227. In some instances, the lactate level data generated by the lactate sensor 27/227 may be used to control one or more operations of the percutaneous circulatory support device 10, such as the flow rate, motor speed of the percutaneous blood pump 50. In some instances, the amount of lactate in the blood that is sensed by the lactate sensor 27/227 is based on an amount of lactate oxidase (LOD) in the blood. In some instances, the amount of lactate in the blood that is sensed by the lactate sensor 27/227 is based on the amount of lactate dehydrogenase (LDH) in the blood. The console 328 may be configured to periodically or continuously collect, record, monitor, save, evaluate and/or display the amount of lactate in the blood.

Additionally, the first display 332 and/or the second display 334 may include a CRT, LED, 3D display, other type of display, for example. The first display 332 and/or the second display 334 may present information relevant to functional and operational parameters of the percutaneous circulatory support system 300, catheter 10 and/or physiological parameters of the patient 316 in a simple format useful to clinicians. For example, the first display 332 and/or the second display 334 may be configure to display information relevant to the lactate levels measured in the blood from the lactate sensor 27/227. The first display 332 and/or the second display 334 may also be configured to display graphical information relevant to the electrical connection of the electrical cable 326 to the console 328, an optical connection to a pressure sensor positioned in the elongate shaft 12 and/or the percutaneous blood pump 50, a power current to the percutaneous blood pump 50, the speed of the impeller of the percutaneous blood pump 50, an arterial pressure, a venous pressure, a power current to the motor, the speed of the motor, rotations per minute measurement of a motor, the voltage driving the motor, the back-EMF from the motor, etc. and any combination or temporal pattern of signals corresponding to one or more of the parameters listed herein. Further, additional parameters (e.g., flow through the percutaneous blood pump 50) may be derived by processing any combination or temporal pattern of signals corresponding to one or more of the parameters listed herein in a time dependent manner. Further, the first display 332 may indicate if the system 300 is operating normally, if the system 300 has detected a specific issue that may require additional (e.g., non-routine) procedures/adjustments, and/or if the system 300 has detected s specific issue that requires emergency procedures. Furthermore, the first display 332 may include information which conveys that a particular response (e.g., action) is needed. For example, the first display 332 may be able to convey information that the percutaneous blood pump 50 has failed and needs to be replaced immediately during a medical procedure.

In some examples, the percutaneous circulatory support system 300 may be designed such that the type and/or arrangement of graphical information displayed on the first display 332 may be different than the information displayed on the second display 334. For example, in some instances, the second display 334 may be designed to display detailed information about one or more functional, operational and/or physiological parameters (e.g., an in-vivo lactate measurement) relating to the circulatory system 300, the catheter 10 and/or the patient 316 while the first display 332 may be designed to provide a simplified, “global” information status summary for one or more functional, operational or physiological parameters relating to the circulatory system 300, the catheter 10 and/or the patient 316.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device comprising:
a catheter including an elongate shaft and a percutaneous blood pump positioned at a distal end of the elongate shaft, the percutaneous blood pump including a blood inlet, a blood outlet, and an impeller assembly positioned therebetween to pump blood from the blood inlet to the blood outlet during use; and
a lactate sensor configured to sense an amount of lactate in the blood.

2. The device of claim 1, wherein the lactate sensor is an electrochemical sensor.

3. The device of claim 1, wherein the amount of lactate is based on an amount of lactate oxidase (LOD) in the blood.

4. The device of claim 1, wherein the amount of lactate is based on an amount of lactate dehydrogenase (LDH) in the blood.

5. The device of claim 1, wherein the lactate sensor is configured to periodically detect the amount of lactate in the blood.

6. The device of claim 1, wherein the lactate sensor is configured to continuously detect the amount of lactate in the blood.

7. The device of claim 1, wherein the lactate sensor is in direct fluid communication with the blood.

8. The device of claim 1, wherein the lactate sensor is located inside of or on an exterior surface of the percutaneous blood pump.

9. The device of claim 8, wherein the lactate sensor is located between the blood inlet and the blood outlet of the percutaneous blood pump.

10. The device of claim 1, wherein the lactate sensor is located inside of or on an exterior surface of the elongate shaft.

11. The device of claim 10, the lactate sensor is located proximal of the percutaneous blood pump.

12. The device of claim 11, wherein the lactate sensor is mounted in a housing on the elongate shaft with the lactate sensor facing an exterior of the elongate shaft.

13. A percutaneous circulatory support system, the system comprising:
a percutaneous circulatory support device including:
a catheter including an elongate shaft and a percutaneous blood pump positioned at a distal end of the elongate shaft, the percutaneous blood pump including a blood inlet, a blood outlet, and an impeller assembly positioned therebetween to pump blood from the blood inlet to the blood outlet during use in a heart of a patient; and
a lactate sensor configured to sense a real-time serum lactate level in the blood of the patient during use of the percutaneous blood pump in the heart of the patient.

14. The system of claim 13, wherein the lactate sensor is secured to an exterior of the elongate shaft proximal of the percutaneous blood pump.

15. The system of claim 14, wherein the lactate sensor is mounted in a housing on the elongate shaft.

16. The system of claim 13, wherein the lactate sensor is positioned between the blood inlet and the blood outlet.

17. The system of claim 13, further comprising an introducer sheath, wherein the introducer sheath includes a lumen configured to deliver the percutaneous blood pump to the heart of the patient, and wherein the lactate sensor is secured to the introducer sheath.

18. An introducer sheath for gaining access to a vasculature of a patient comprising:
a hub including a hemostasis valve;
an elongate shaft extending distally from the hub, the elongate shaft including a lumen extending therethrough; and
a lactate sensor configured to sense an amount of lactate in the blood.

19. The introducer sheath of claim 18, wherein the lactate sensor is mounted on a distal end region of the elongate shaft.

20. The introducer sheath of claim 18, wherein the lactate sensor is movably postionable through a passageway of the hub.
